(19) United States
(12) Patent Application Publication
(10) Pub. No.: US 2014/0371242 A1
(43) Pub. Date: Dec. 18, 2014

(54) Azathioprine Oral Suspensions and Methods of Use

(71) Applicant: Tsu-I Catherine Wang, Sugar Land, TX (US)

(72) Inventor: Tsu-I Catherine Wang, Sugar Land, TX (US)

(73) Assignee: PROFESSIONAL COMPOUNDING CENTERS OF AMERICA, Houston, TX (US)

(21) Appl. No.: 13/918,343

(22) Filed: Jun. 14, 2013

(51) Int. Cl. A61K 31/52

(52) U.S. Cl. CPC A61K 31/52
USPC 514/263.2

(57) ABSTRACT

Compositions of azathioprine oral suspensions are disclosed. Disclosed azathioprine oral suspensions may be used to administer azathioprine to subjects such as children and geriatric patients that may have difficulty in swallowing solid dosage forms. The disclosed azathioprine oral suspension may be used for treating autoimmune diseases such as rheumatoid arthritis, pemphigus, Behcet's disease, autoimmune hepatitis, and inflammatory bowel disease, among others. According to an embodiment, an aqueous or non-aqueous vehicle may be used for azathioprine oral suspension. According to a different embodiment, sugar free azathioprine oral suspension may also be produced.

### CROSS-REFERENCE TO RELATED APPLICATIONS

N/A

### BACKGROUND

1. Field of the Disclosure

The present disclosure relates in general to therapeutic formulations, and more particularly, to an oral suspension for treating autoimmune diseases.

2. Background Information

An autoimmune disease is a condition where the immune system attacks both unhealthy and healthy cells in the human body. This may affect several parts of the body; furthermore, autoimmune responses are very common in organ and tissue transplants. There are more than 50 types of autoimmune diseases, most of them with similar symptoms. This makes selecting the treatment method a very hard task for health care providers.

Azathioprine is an immunosuppressive drug used in autoimmune diseases with difficult treatment. Furthermore, azathioprine is used in combination with other immunosuppressive therapy agents in order to treat rejection in organ and tissue transplantations. Sometimes the body tries to reject new donor tissue and azathioprine helps to prevent this rejection by suppressing the body's immune or defense system. Azathioprine is also used in some auto-immune illnesses, such as rheumatoid arthritis, where the body has an overactive immune response against itself. Azathioprine may also be used to treat diseases such as pemphigus, Behcet's disease, autoimmune hepatitis, inflammatory bowel disease, and Crohn's disease, among others.

Azathioprine is usually administered orally, which results a challenge when treating children or geriatric patients who exhibit difficulty in swallowing solid oral preparations, such as tablets.

For the aforementioned reasons, there is a need for a new administration form for azathioprine that may allow the administration of azathioprine to children and geriatric patients.

### SUMMARY

The present disclosure describes an azathioprine oral suspension that may be administered to children or geriatric patients in need of azathioprine and who may be unable to swallow solid dosage forms. The disclosed azathioprine oral suspension may be administered as an oral suspension for treating autoimmune diseases and to patients in need of post-surgical treatment for an organ or tissue transplant. More specifically, the disclosed oral suspension may be used for treating autoimmune diseases such as rheumatoid arthritis, pemphigus, Behcet's disease, autoimmune hepatitis, inflammatory bowel disease, and Crohn's disease, among others. According to an embodiment, azathioprine oral suspension may include a concentration of between about 5 mg/ml to about 50 mg/ml, most suitable concentration may be of about 50 mg/ml. Furthermore, azathioprine within the disclosed oral suspension may be administered at a dosage ranging from about 0.5 mg/Kg/day to about 2.5 mg/Kg/day, most suitable dosage may be of about 25-100 mg/day. According to an embodiment, an aqueous or non-aqueous vehicle may be included in azathioprine oral suspension. According to a different embodiment, sugar free azathioprine oral suspension may also be produced.

It is therefore an object of the present disclosure to provide an oral suspension composition for treating autoimmune diseases in children and geriatric patients. Additional aspects of the disclosure, together with the advantages and novel features appurtenant thereto, will be set forth in part in the description that follows, and in part will become apparent to those skilled in the art upon examination of the following, or may be learned from the practice of the disclosure. The objects and advantages of the disclosure may be realized and attained by means, instrumentalities, and combinations particular pointed out in the appended claims.

### DETAILED DESCRIPTION

The present disclosure is here described in detail with reference to embodiments, which form a part here. Other embodiments may be used and/or other changes may be made without departing from the spirit or scope of the present disclosure. The illustrative embodiments described in the detailed description are not meant to be limiting of the subject matter presented here.

### Definitions

As used here, the following terms may have the following definitions:

“Active pharmaceutical ingredient (API)” refers to a substance that induces a suitable pharmacological or physiological effect, and may include agents with therapeutic, prophylactic, or cosmeceutical effects.

“Treating” and “treatment” refers to a reduction in the severity and/or frequency of symptoms, elimination of symptoms and/or underlying cause, and improvement or remediation of damage.

### Description

The present disclosure may relate to a composition of ingredients that, in one embodiment may be an azathioprine oral suspension. The disclosed azathioprine oral suspension may include a combination of suitable vehicles and azathioprine as active pharmaceutical ingredient (API). The disclosed azathioprine oral suspension may allow the administration of azathioprine to children and geriatric patients that may have difficulty in swallowing solid dosage forms

### Azathioprine Oral Suspension Composition

According to a suitable embodiment, azathioprine present in the disclosed azathioprine oral suspension may include a concentration of between about 5 mg/mI to about 50 mg/ml, most suitable concentration may be of about 50 mg/ml. The disclosed azathioprine oral suspension may be administered at a dosage ranging from about 0.5 mg/Kg/day to about 2.5 mg/Kg/day, most suitable dosage may be of about 25-100 mg/day. According to an embodiment, an aqueous or non-aqueous vehicle may be included in azathioprine oral suspension. According to a different embodiment, sugar free azathioprine oral suspension may also be produced.

The disclosed composition may be administered as an oral suspension to a patient in need of post-surgical treatment for an organ or tissue transplant. Furthermore, the disclosed oral suspension may be administered for treating autoimmune diseases such as rheumatoid arthritis, pemphigus, Behcet's disease, autoimmune hepatitis, Crohn's disease, and inflammatory bowel disease, among others.

Additionally, Azathioprine may be used in systemic lupus erythematosus patients who require a maintenance dose of 15 mg or higher of prednisone and those who experience recurrent flares. Azathioprine is used as an adjuvant in the oral steroid therapy of pemphigus and myasthenia gravis, as a “steroid-sparing” agent for reducing the dose of cortisteroids. Furthermore, azathioprine was shown to be very effective in eczema and atopic dermatitis in researches, even though it is not commonly used.

An azathioprine oral suspension may include more benefits and be more effective compared to solid oral dosage forms and injected applications. For example, oral suspensions may be critically important for patients (infants, children, and geriatric patients) who are unable to swallow solid dosage forms. Additionally, oral suspension forms exhibit faster API dissolution and absorption rate than solid dosage forms. Therefore, an azathioprine oral suspension may open new perspectives for the treatment of the aforementioned diseases.

### Azathioprine

Azathioprine is an immunosuppressant that may be used with other immunosuppressive medications to prevent organ rejection. Furthermore, azathioprine is often prescribed in order for doses from other immunosuppressant medications to be decreased, this may allow reducing side effects. Azathioprine is metabolized to 6-mercaptopurine, which interferes with purine synthesis and thus blocks new DNA synthesis necessary for rapidly dividing cells, i.e., it nonspecifically decreases proliferation of rapidly dividing cells. Side effects include thrombocytopenia, leukopenia, megaloblastic anemia, pancreatitis, and hepatitis. Azathioprine is usually administered in oral solid dosage forms (i.e. in tablets) or may be injected.

### Non-Aqueous Vehicles

Non-aqueous vehicles for azathioprine oral suspension may include, but is not limited to, almond oil, Silica Gel (as a dispersing or thickening agent), butylated hydroxytoluene (as antioxidant), and medium chain triglycerides. Furthermore, azathioprine oral suspension may include other vegetable oils such as almond oil, corn oil, olive oil, peanut oil, sesame oil, and soybean oil, among others. These oils may include other suitable ingredients such as antioxidants, wetting agents, surfactants, dispersing agents, thickening agents, flavors, food colors, preservatives, among others.

### Aqueous Vehicles

Aqueous vehicles for azathioprine oral suspension may include suitable agents such as sucrose (as sweetener), citric acid (as pH adjuster) , sodium benzoate (as preservative). Additionally, azathioprine oral suspension may include an oral suspending vehicle. This oral suspending vehicle may include, but is not limited to, methylparaben (as preservative), propylparaben (as preservative), potassium sorbate (as preservative), citric acid (buffer/PH adjuster), sodium phosphate dibasic (buffer/pH adjuster), microcrystalline cellulose (as dispersing agent), carboxymethylcellulose sodium (as suspending agent/thickening agent), xanthan gum (as suspending/thickening agent), carrageenan (as suspending/thickening agent), simethicone (as anti-foaming agent), water. Furthermore, azathioprine oral suspension may include a sugar free syrup vehicle. This sugar free syrup vehicle may include, but is not limited to, methylparaben (as preservative), propylparaben (as preservative), glycerin (as co-solvent), sorbitol (as sweetener), saccharin sodium (as sweetener), citric acid (buffer/pH adjuster), sodium citrate (buffer/pH adjuster), potassium sorbate (as preservative), food flavor, food color and water.

### Manufacturing Method for Azathioprine Oral Suspension

According to an embodiment, Azathioprine may be mixed with other solid ingredients, followed by mixing with the wetting agent to form a paste. A suitable vehicle base can then be mixed in. Flavors and coloring agents may also be added tot he mix.

### EXAMPLES

Example #1 is an application for azathioprine oral suspension, where azathioprine oral suspension may be used to treat post-surgical patients from organ transplantation procedures such as liver transplants, kidney transplants, heart transplants, intestine transplants, and lung transplants, among others.

Example #2 is an application for azathioprine oral suspension, where azathioprine oral suspension may be used to treat post-surgical patients from tissue transplantation procedures such as musculoskeletal transplants, skin transplants, heart valves transplants and cornea transplants, among others.

What is claimed is:

1. A pharmaceutical composition for oral administration comprising azathioprine in a concentration of about 5 mg/10 ml to about 50 mg/10 ml.

2. A composition according to claim 1, which is in the form of an oral suspension.

3. A composition according to claim 2, further comprising one selected from the group consisting of almond oil, corn oil, olive oil, peanut oil, sesame oil, soybean oil, and combinations thereof.

4. A composition according to claim 1, which is in the form of a tablet.

5. A composition according to claim 2, further comprising one selected from the group consisting of antioxidants, wetting agents, surfactants, dispersing agents, thickening agents, flavors, food colors, preservatives, and combinations thereof.

6. A method of treating an autoimmune disease, comprising orally delivering to a patient an effective amount of a pharmaceutical composition that comprises azathioprine.

7. The method according to claim 6, wherein the autoimmune disease is selected from the group consisting of rheumatoid arthritis, pemphigus, Behcet's disease, autoimmune hepatitis, inflammatory bowel disease, and Crohn's disease.

8. The method according to claim 6, wherein the azathioprine is present in a concentration of about 5 mg/ml to about 50 mg/ml.

9. The method according to claim 6, wherein the pharmaceutical composition is administered in multiple doses per day.

10. The method according to claim 6, wherein the pharmaceutical composition is administered to provide azathioprine at a dosage of about 0.5 mg/Kg/day to about 2.5 mg/Kg/day.

11. The method according to claim 6, wherein the pharmaceutical composition is administered to provide azathioprine at a dosage of about 25-100 mg/day.

12. The method according to claim 6, wherein the pharmaceutical composition further comprises a non-aqueous vehicle.

13. The method according to claim 6, wherein the non-aqueous vehicle is selected from the group consisting of almond oil, silica gel, butylated hydroxytoluene, at least one medium chain triglyceride, and combinations thereof.

14. The method according to claim 6, wherein the pharmaceutical composition further comprises an aqueous vehicle.

15. The method according to claim 6, wherein the pharmaceutical composition is sugar free.

16. The method according to claim 6, wherein the pharmaceutical composition comprises at least one selected form the group consisting of methylparaben, propylparaben, glycerin, sorbitol, saccharin sodium, citric acid, sodium citrate, potassium sorbate, food flavor, food color, water, and combinations thereos.

17. The method according to claim 6, wherein the pharmaceutical composition further comprises at least one suspending vehicle selected from the group consisting of methylparaben, propylparaben, potassium sorbate, citric acid, sodium phosphate dibasic, microcrystalline cellulose, carboxymethylcellulose sodium, xanthan gum, simethicone, water, and combinations thereof.
